(19) United States
(12) Patent Application Publication
(10) Pub. No.: US 2010/0086651 A1
(43) Pub. Date: Apr. 8, 2010

(54) LARGE COACERVATED CAPSULES

(76) Inventor: Grégory Dardelle, Jonzier-Epagny (FR)

Correspondence Address: WINSTON & STRAWN LLP;PATENT DEPARTMENT, 1700 K STREET, N.W., WASHINGTON, DC, 20006, US

(21) Appl. No.: 12/597,091

(22) PCT Filed: May 20, 2008
(86) PCT No.: PCT/IB08/51970

§ 371 Date: Oct. 22, 2009

(30) Foreign Application Priority Data
May 21, 2007 (EP) 07108563.3

(51) Int. Cl. B32B 5/16; C09K 3/00; A23L 1/226; A61K 8/11

(52) U.S. Cl. 426/89; 428/402; 252/182.12; 512/4

(57) ABSTRACT

The invention relates to a coacervated capsule comprising from 10 to 95% by weight of the capsule of a core comprising essentially a hydrophobic material, and from 90 to 5% by weight of the capsule of a coating layer comprising essentially a protein, and optionally a non-protein polymer, wherein the core further comprises from 0.01% to 30% by weight of the capsule of a cellulose ether derivative having an alkoxyl content from 35 to 60% and a degree of substitution of alkoxyl groups per anhydroglucose unit of from 2 to 3 and the viscosity of the core, measured at ambient temperature, is from about 100 mPa·s to 30000 mPa·s.

### TECHNICAL FIELD

The present invention relates to large coacervated capsules for use as a delivery system and particularly relates to coacervated capsules comprising ethyl cellulose in the core of the capsules for use in the flavour and fragrance industry.

### BACKGROUND ART

Coacervation, also called aqueous phase separation, is a very well known technique for encapsulating hydrophobic liquids. The process provides oil-containing microcapsules, the encapsulating material being a gelled hydrophilic colloid that is impervious to the oil and deposited evenly and densely around the oil. The encapsulating material is a protein which may be complexed with another colloid having an opposite electric charge.

A coacervation process may be “simple” or “complex”. The former designation is employed when a single protein is used to form a capsule wall as phase separation is taking place. The latter term designates the use of a second oppositely charged non-protein polymer to bring about phase separation. Complex coacervation method is widely practiced in commercial processes and has been well described in the literature. In particular U.S. Pat. No. 2,800,457 and U.S. Pat. No. 2,800,458 disclose complex coacervation in a very detailed manner.

Generally, a coacervation process comprises four basic steps consisting of, respectively, emulsification, phase separation, wall formation and wall hardening. In a complex coacervation process the wall surrounding the core material is, as mentioned above, constituted of two oppositely charged high molecular weight colloids. In most of the cases, the positively charged colloid used is a gelatin, a functional protein derived from collagen by hydrolysis and subsequent extraction.

One advantage of coacervation as a technique is considered to be the very small size of the particles enabling them to be used in products where it is undesirable to have visible capsules. Typically the capsules have a diameter of from 100 to 300 microns (as described in U.S. Pat. No. 5,759,599).

In U.S. Pat. No. 5,051,305, coacervated microcapsules comprising very small droplets of perfume oil are stabilized by providing ethyl cellulose dissolved in the perfume oil. The only reference to the particle size is in the example, where it is stated that the average oil drop diameter is from 25 to 50 microns. According to column 2 lines 44 to 45 of this document, the ethyl cellulose ideally has a viscosity of between 6 and 8 centipoise.

In EP-A2-1,533,364, capsule particles are disclosed that vary in diameter from about 10 nanometers to about 1000 microns, the most preferred range being from about 2 to about 15 microns. The core of the capsules typically comprises a fragrance and a solvent, the solvent providing stability to the fragrance. All of the examples refer to interfacial polymerization techniques, rather than coacervation. Any reference to ethyl cellulose is general in nature and gives no disclosure or teaching of the class of cellulose ether derivatives required by the present invention order to be able to provide large coacervated capsules.

FR 1279236 and FR 1279239 both disclose processes for preparing coacervated capsules by forming an oil-in-water emulsion comprising a hydrophobic material and adding a thickening agent, methylcellulose, to the aqueous phase. Coacervation of the thickened emulsion is then performed. The methyl cellulose is water soluble, rather than oil soluble and so will not have the desired effect on an oil phase. There is no disclosure or teaching of thickening an oily phase to achieve the size of coacervated particles required by the present invention.

Nevertheless, there is a clear demand for capsules which are visible to the naked eye. For instance, in certain consumer products vary large capsules can be used to enhance the aesthetic appeal of the product or to provide a visual cue with which a benefit can be associated.

Thus, it would be desirable to be able to provide very large coacervated capsules.

In “ Optimisation d'une method de microencapsulation par separation de phases. Rôle de 1′hydroxypropylméthylcellulose comme agent nucléaire”, V. Kaltsatos, M. Rollet, J. Perez S.T.P. PHARMA, vol. 5, No. 2, 1989, p96-102, coacervated particles having a diameter from 800 μm to 2000 μm are disclosed. The capsule walls are based entirely on ethyl cellulose. This is entirely different from using ethyl cellulose in a core to thicken an oily phase and then providing a separate coacervate shell around such a core.

### SUMMARY OF THE INVENTION

Accordingly the present invention provides a coacervated capsule comprising:
(a) from 10 to 95% by weight of the capsule of a core comprising essentially a hydrophobic material, and
(b) from 90 to 5% by weight of the capsule of a coating layer comprising essentially a protein, and optionally a non-protein polymer
   wherein the core further comprises from 0.01% to 30% by weight of the capsule of a cellulose ether derivative having an alkoxyl content from 35 to 60% and a degree of substitution of alkoxyl groups per anhydroglucose unit of from 2 to 3, and the viscosity of the core, measured at ambient temperature, is within the range of from about 100 mPa·s to 30,000 mPa·s.

Surprisingly, the combination of the cellulose ether derivative and the hydrophobic material in the core enables capsules having a diameter of more than 1000 microns to be stably prepared.

The invention further provides a process for the preparation of coacervated capsules comprising the step of:
(a) preparing a core mixture comprising:
   (i) a hydrophobic material, and
   (ii) a cellulose ether derivative having an alkoxyl content from 35 to 60% and a degree of substitution of alkoxyl groups per anhydroglucose unit of from 2 to 3,
      the core mixture having a viscosity, measured at ambient temperature, of from about 100 mPa·s to 30,000 mPa·s;
(b) providing an aqueous solution comprising a protein and, optionally a non-protein;
(c) mixing the core mixture and the aqueous solution to form an emulsion or suspension; and
(d) inducing phase separation such that the protein and, optionally a non-protein polymer, form a wall around the core mixture.

The invention also provides the use of a cellulose ether derivative having an alkoxyl content from 35 to 60% and a degree of substitution of alkoxyl groups per anhydroglucose unit of from 2 to 3 in the hydrophobic core of coacervated capsules to provide capsules having an average size of more than 1000 microns.

### BRIEF DESCRIPTION OF THE DRAWINGS

FIGS. 1a and 1b show coacervated capsules according to the invention;

FIG. 2 shows coacervated capsules prepared without the cellulose ether derivative in the core component.

### DETAILED DESCRIPTION

Core Material

The core of the coacervated capsule comprises a hydrophobic material. It may be in the liquid or solid state. Preferably, it is liquid. Hydrophobic materials are generally regarded as materials that are not miscible in water at 25° C. and, when added to it, form a separate, hydrophobic phase. For the purpose of the present invention, the term hydrophobic material includes material that is in the solid state at the temperatures generally employed in coacervation processes, that is, at less than or equal to about 50° C. Such solid material may be present in the form of crystals, for example. Preferably, if the solid material is liquefied by heating above its melting point, it forms a separate phase in water at that temperature.

Preferred hydrophobic materials include flavours, fragrances, fats, oils, mouth-feel enhancers, nutraceuticals, drugs, other bioactive ingredients or mixtures thereof. Flavours and fragrances are particularly preferred.

The terms “flavours” and “fragrances” as used herein are deemed to define a variety of flavour and fragrance materials of both natural and synthetic origins. They include single compounds or mixtures. Specific examples of such components may be found in the literature, e.g. in Fenaroli's Handbook of Flavor Ingredients, 1975, CRC Press; synthetic Food Adjuncts, 1947 by M. B. Jacobs, edited by van Nostrand; or Perfume and Flavor Chemicals by S. Arctander 1969, Montclair, N.J. (USA). These substances are well known to the person skilled in the art of perfuming, flavouring and/or aromatising consumer products, i.e. of imparting an odour and/or flavour or taste to a consumer product traditionally perfumed or flavoured, or of modifying the odour and/or taste of the consumer product.

Nutraceuticals are edible materials such as foods or food ingredients that provide medical or health benefit to a human or animal individual upon consumption. Nutraceuticals include, for example, polyunsaturated fatty acids and/or oils comprising them, vitamins, minerals, co-enzyme Q, carnitine, botanical extracts, for example from ginseng, ginko biloba, Saint John's Wort, Saw Palmetto, functional foods such as oat, bran, psyllium, lignins, prebiotics, canola oil and stanols, for example. Preferably, the hydrophobic material comprises flavours and/or fragrances. Many bioactive principles, but in particular flavours and/or fragrances, or compositions of flavours and/or fragrances, have a high proportion of volatile compounds and/or components.

The hydrophobic material preferably comprises at least 5 wt. %, more preferably at least 10 wt. %, even preferably at least 20 wt. %, most preferably at least 30 wt. %, e.g. at least 40 wt. % of chemical compounds having a vapour pressure of ≧0.007 Pa at 25° C.

Preferably, at least 10 wt. % have a vapour pressure of ≧0.1, more preferably, at least 10 wt. % have a vapour pressure of ≧1 Pa at 25° C., and most preferably, at least 10 wt. % have a vapour pressure of ≧10 Pa at 25° C. The value of ≧0.007 Pa at 25° C. is selected because it encompasses most of the compounds used by the skilled flavourist and/or perfumer. Compounds meeting these criteria are generally regarded as having a volatile character. In addition, compounds that remain odourless due to a lower volatility are excluded. The limit of 10 wt % of such compounds is regarded to constitute a substantial part of the ingredient. The method of the present invention, however, allows for efficient encapsulation of more volatile ingredients being present in higher amounts of the total ingredients.

For the purpose of the present invention, the vapour pressure is determined by calculation using the method disclosed in “EPI suite”; 2000 U.S. Environmental Protection Agency.

The fragrance compound limonene is adduced for illustrating the determination of vapour pressure by calculation: by applying the method “EPI suite”, limonene is calculated to have a vapour pressure of about 193 Pa at 25° C.

A cellulose ether derivative is present as part of the hydrophobic core material. Preferably the cellulose ether derivative is a hydroxy C₁ to C₄ alkyl ether cellulose. Particularly preferred are methyl cellulose, ethyl cellulose, hydroxymethyl cellulose, hydroxyethyl cellulose, hydroxypropyl methyl cellulose or mixtures thereof. Especially preferred are ethyl cellulose or hydroxypropyl cellulose. Most preferred is ethyl cellulose.

The presence of the cellulose ether derivative enables the formation of capsules which have a much larger diameter than traditionally associated with protein-based coacervated capsules.

It is believed that this occurs because the cellulose ether derivative effectively prevents the breaking and coalescence of the core droplets during the coacervation process such that much larger droplet are obtainable.

The alkoxyl content of the modified cellulose ether is from 35 to 60%, more preferably from 40 to 55%, most preferably from 45 to 52.5%.

The degree of substitution of alkoxyl groups per anhydroglucose unit is of from 2 to 3, preferably from 2.22 to 2.81.

The viscosity, measured at ambient temperature, of the core droplet containing the modified cellulose ether is from about 100 mPa·s to 30,000 mPa·s. More preferably the viscosity is from 200 mPa·s to 25,000 mPa·s, even more preferably from 250 mPa·s to 15,000 mPa·s, most preferably from 300 mPa·s to 10,000 mPa·s.

Below a viscosity of 100 MPa·s of the core droplet, it is found that the particle size of the coacervated capsule is very significantly below that required by the present invention.

In order to achieve the correct viscosity of the core droplet, it is preferred that the viscosity of the modified cellulose ether is from 50 mPa·s to 1,000 mPa·s, more preferably 75 mPa·s to 750 mPa·s, most preferably 100 mPa·s to 500 mPa·s, measured as a 5% solution based on 80% toluene 20% ethanol, at 25° C. in an Ubbelohde viscometer.

The molecular weight of the cellulose ether derivative is preferably within the range of from 50,000 to 2,000,000, more preferably from 75,000 to 1,500,000, most preferably from 100,000 to 1,250,000.

Commercially available cellulose ether derivatives include, for instance, the Klucel® and Aqualon® ranges, ex Hercules.

The amount of cellulose ether derivative is preferably within the range of from 0.01 to 30%, more preferably from 0.05 to 15%, even more preferably 0.1 to 8%, most preferably 0.5 to 6%, e.g. 2.5 to 4.5% by weight based on the total weight of the core material.

It has been found that within this range the cellulose ether derivative provides excellent integrity of the core droplets for production of the large coacervated capsules.

The core droplets of hydrophobic material have an average diameter of more than 1000 μm, preferably more than 1200 μm, more preferably more than 1300 μm, and most preferably more than 1400 μm. Average refers to the arithmetic mean. For the sake of simplicity, the diameter of the emulsified droplets or the suspended particles is taken as the size of the microcapsules of the present invention.

The droplets form the hydrophobic phase of an emulsion during the coacervation process. To form the emulsion, any suitable process known to the skilled person for performing the emulsification can be used. For example, preparation using a 4-bladed impeller shearing device operated at 300s⁻¹, the ratio of the diameter of the container to the diameter of the blades being about 2:1, would be suitable for the purposes of the present invention. Nevertheless, in order to ensure that larger droplets are maintained throughout the process it is highly desirable to shear with the minimum energy in order to avoid unnecessary fragmentation of the droplets.

Coating Layer

The coating layer comprises a protein and, optionally, a non-protein polymer and forms a coacervate around the hydrophobic droplet. Preferably, the non-protein polymer is charged oppositely to the protein.

These materials are also referred to commonly as hydrocolloids, that is polymeric substances that can be dissolved in water, optionally at elevated temperatures, e.g. up to 90° C. These encompass polymers such as proteins, polysaccharides and polyacids, for example, that are generally known to be useful in coacervation methods.

The present invention encompasses “simple” and “complex” coacervation. In simple coacervation, protein alone is used to form a capsule wall as phase separation is taking place. Complex coacervation refers to methods in which a generally oppositely charged non-protein polymer and a protein polymer together form the capsule wall. According to the principles of complex coacervation the present invention method provides the optional addition of an oppositely charged non-protein polymer, preferably a polysaccharide, to the hydrocolloid solution.

Proteins useful in coacervation processes include albumins, vegetable globulins and gelatines. The molecular weight of the protein is typically in the order of 40,000 to 500,000 preferably 20,000 to 250,000. Some protein aggregates, however, may have molecular weights even greater than this.

Preferably, the protein is a gelatine. It is preferable to use gelatine having good physicochemical and chemical properties as typified by good film forming ability, amphoteric properties, the controllability of the quantity of charges by pH, and, preferably, the occurrence of the change from solution to gel at a critical temperature. Stated specifically, any gelatine that satisfies the specification for use in production of microcapsules may be employed.

The gelatine may be fish, pork, beef, and/or poultry gelatine, for example. According to a preferred embodiment, the protein is fish, beef or poultry gelatine. According to a more preferred embodiment, the protein is warm water fish gelatine. Preferably, the warm water fish gelatine has a bloom of from about 150 to about 300 bloom, more preferably from about 200 to about 300 bloom. Preferably, the warm water fish gelatine has ≧250 bloom. According to the general knowledge, warm water fish are fish that are capable of tolerating water above 27° C. over prolonged time.

Typical non-protein polymers useful in complex coacervation methods include, in particular, negatively charged polymers. For example, they may be selected from gum arabic, xanthan, agar, alginate salts, cellulose derivatives, for example carboxymethyl cellulose, pectinate salts, carrageenan, polyacrylic and methacrylic acid, and/or mixtures thereof. Further suitable non-proteins can be derived from the literature, for example from WO 2004/022221, page 4, lines 27-29.

The protein and, optionally, non-protein polymers are usually dissolved in water to form a hydrocolloid solution. Preferably, in the aqueous hydrocolloid solution, the protein is present in an amount of from 0.5 to 3.5 wt %, more preferably from 1 to 2 wt %.

If present, the amount of polysaccharide is preferably from 0.5 to 3.5 wt %, more preferably from 1 to 2% wt. % in the aqueous solution.

In a particular embodiment, the weight ratio between the protein and the non-protein polymer is from about 3:1 to 1:3, more preferably 2:1 to 1:1, most preferably about 3:2.

Cross-Linking Agent

A cross-linking agent is typically used to harden the coating layer. Suitable cross-linking agents include formaldehyde, acetaldehyde, glutaraldehyde, glyoxal, chrome alum, or transglutaminase. Preferably, transglutaminase is used at 10-100, preferably 30-60 activity units per gram of gelatine. This enzyme is well described and commercially obtainable.

Preparation

According to a preferred embodiment, the coacervated capsule is prepared by forming a first solution of the protein material above its gelling temperature and a second aqueous solution of the non-protein polymer. The two solutions are mixed to form a third solution.

The hydrophobic core material is then mixed with the cellulose ether derivative and introduced into the third solution under shear to form an emulsion or suspension. The emulsion and/or suspension may be prepared in a conventional manner Preferably, the hydrophobic material comprising the cellulose ether derivative is slowly added during 3-10 min, preferably 4-6 min, with a stirrer being adjusted to 300-400 rpm. The stirrer speed can be adjusted as desired.

In an alternative embodiment, the emulsion can be prepared by membrane emulsification. Typically this involves passing the hydrophobic core material through a membrane having the desired pore size into a solution comprising the protein material and, optionally, the non-protein polymer and then vibrating the resulting mixture until an emulsion forms. The advantage of this process is that a narrower range of particle sizes is achievable than when standard emulsification techniques are used.

In the next step, the phase separation is induced and a coacervate phase is created. The coacervate phase is generally based on the protein and, optionally, the non-polymer compound. This step is also referred to as phase separation. This step may be preferably accomplished by modifying, preferably lowering, the pH to below the iso-electric point of the protein. If a non-protein polymer is present, the pH is preferably adjusted so that the positive charges on the proteins are neutralized by the negative charges on the non-protein polymer.

Phase separation may be induced by various other ways, in general by changing the physico-chemical environment of the solution. Depending on the kind of coacervation process (simple; complex) different ways of inducing phase separation can be applied, e.g. salting out or addition of a second high molecular weight component so as to induce entropic phase separation.

The temperature of the mixture is then reduced to below the gelling temperature of the protein. The determination of the gelling temperature of the gellable protein, preferably gelatine, is established, in part by experiment, the techniques of which are well known in the art.

In the final step, cross-linking is performed to harden the colloid wall comprising the protein around droplets of the hydrophobic material present in an emulsion and/or suspension. This step takes place spontaneously once the step of formation of a coacervate phase is induced.

Cross-linking is typically conducted at a temperature within the range of 5 to 40° C. Similarly, the pH during the cross-linking step is preferably adjusted to a level at which cross-linking can effectively be conducted. For example, if cross-linking is catalysed by the action of transglutaminase, the pH may preferably be adjusted to 3-7, preferably 3.5-5.5.

End Products

The microcapsules according to the present invention can be used in many kinds of applications or consumer end products where capsules visible to the naked eye are desired. Particular fields of interest are flavours and fragrances. Therefore, perfuming or flavouring compositions comprising microcapsules according to the invention, optionally together with other perfuming or flavouring co-ingredients, are also aspects of the present invention.

The invention is particularly useful where visual cues are desired to inform and reassure the consumer that a particular encapsulated product is being delivered, e.g. in oral care products such as toothpastes and chewing gums.

Suitable end products also include household care products, such as detergents. A further group of products where the invention can be applied is personal care products, such as cosmetics and shampoos.

### EXAMPLES

The invention will now be described in a more detailed manner in the example below, wherein the temperatures are indicated in degrees Celsius and the abbreviations have the usual meaning in the art.

### Example 1

Preparation of Capsules According to the Invention

Limonene was microencapsulated within a hydrocolloid shell according to a complex coacervation process. Warm water fish gelatine (200 Bloom, supplied by Weishardt) and gum Arabic (Efficacia®, from CNI) were used as the hydrocolloids. A stock solution of gelatine (solution A) was prepared by mixing 180 g of warm deionised water and 20 g of gelatine in a vessel until completely dissolved; the solution was then maintained at 40° C. A stock solution of gum Arabic (solution B) was prepared by mixing 180 g of cold deionised water and 20 g of gum Arabic in a vessel until completely dissolved; the solution was then warmed and kept at 40° C. A solution of limonene (solution C) was prepared by adding 5 g of ethyl-cellulose (ECT300—Hercules) in 95 g of limonene in a vessel until completely dissolved. 105.4 g of solution A was mixed with 70.3g of solution B in a vessel under gentle agitation (the gelatine/gum Arabic ratio is 1.5:1). The pH was adjusted to 4.6 with a 50% w/w aqueous lactic solution. 70.3 g of solution C was slowly added to this mixture and homogenised with a stirrer at 150 RPM during 5 min to reach an average droplet size of between 1000 and 1500 μm.

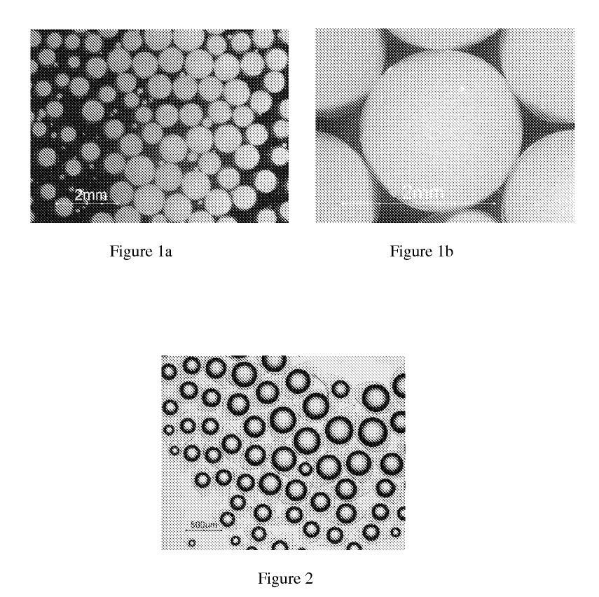

The system was then diluted by the addition of 354.1 g of warm deionised water, bringing the total hydrocolloid concentration to 3.4% w/w. The mixture was finally cooled to 20° C. at a rate of 0.5° C.min⁻¹. The stirring speed was slightly decreased, the pH adjusted to 4.5 and 4.22 g of transglutaminase (ACTIVA® WM, ex Ajinomoto) was added. Cross-linking was allowed to proceed overnight at 20° C. The result is an aqueous suspension of large coacervated capsules, as can be seen in FIGS. 1a and 1b of the drawings.

The experiment was repeated except that the ethyl cellulose was excluded. The results are shown in FIG. 2.

1.-12. (canceled)

13. A process for the preparation of coacervated capsules which comprises:
(a) preparing a core mixture comprising:
(i) a hydrophobic material, and
(ii) a cellulose ether derivative having an alkoxyl content from 35 to 60% and a degree of substitution of alkoxyl groups per anhydroglucose unit of from 2 to 3,
with the core mixture having a viscosity, measured at ambient temperature, of from about 100 mPa·s to 30,000 mPa·s;
(b) providing an aqueous solution comprising a protein and, optionally a non-protein;
(c) mixing the core mixture and the aqueous solution to form an emulsion or suspension; and
(d) inducing phase separation such that the protein and, optionally a non-protein polymer, form a wall around the core mixture.

14. A process according to claim 13 wherein the capsules have an average particle size of greater than 1000 microns.

15. Capsules obtainable from the process of claim 14.

16. A coacervated capsule comprising:
(a) from 10 to 95% by weight of the capsule of a core comprising essentially a hydrophobic material, and
(b) from 90 to 5% by weight of the capsule of a coating layer comprising essentially a protein, and optionally a non-protein polymer,
wherein the core further comprises from 0.01% to 30% by weight of the capsule of a cellulose ether derivative having an alkoxyl content from 35 to 60% and a degree of substitution of alkoxyl groups per anhydroglucose unit of from 2 to 3 and the viscosity of the core, measured at ambient temperature, is from about 100 mPa·s to 30,000 mPa·s.

17. A capsule according to claim 16, wherein the cellulose ether derivative is an optionally substituted C1 to C4 alkyl cellulose.

18. A capsule according to claim 17, wherein the alkyl cellulose is hydroxypropyl methyl cellulose or ethyl cellulose.

19. A capsule according to claim 18, wherein the alkyl cellulose is ethyl cellulose.

20. A capsule according to claim 16, wherein the degree of substitution of the cellulose ether derivative is from 2.22 to 2.81.

21. A capsule according to claim 16, wherein the molecular weight of the cellulose ether derivative is from 50,000 to 2,000,000.

22. A capsule according to claim 16, wherein the hydrophobic material is a flavor or a fragrance.

23. A capsule according to claim 16 wherein the average particle size of the capsules is greater than 1000 microns.

24. A plurality of capsules according to claim 16.
